(19) United States
(12) Patent Application Publication
(10) Pub. No.: US 2026/0325030 A1
(43) Pub. Date: Oct. 22, 2026

(54) SYSTEM FOR SENSING STATE OF AN OBJECT IN AN ENVIRONMENT

(71) Applicants: WOLFGANG RICHTER, Vancouver (CA); Faranak Zadeh, Vancouver (CA)

(72) Inventors: WOLFGANG RICHTER, Vancouver (CA); Faranak Zadeh, Vancouver (CA)

(21) Appl. No.: 19/428,307

(22) Filed: Dec. 22, 2025

Related U.S. Application Data
(63) Continuation of application No. 17/192,675, filed on Mar. 4, 2021, now abandoned.
(60) Provisional application No. 62/985,328, filed on Mar. 5, 2020.

(51) Int. Cl. A61B 5/268; A61B 5/00

(52) U.S. Cl. CPC A61B 5/268; A61B 5/0022; A61B 5/685; A61B 2562/0233; A61B 2562/08

(57) ABSTRACT

Disclosed is a system for sensing state of an object in an environment. The system includes a generator, an emitting electrode, a patch, and an electronic circuitry. The generator generates an alternating electric field. The emitting electrode emits the alternating electric field on the patch, the object and the electronic circuitry. The patch is having a top surface and a bottom surface. The patch includes a plurality of top markers, plurality of top control electrodes, plurality of bottom markers, plurality of bottom control electrodes, and a collector electrode. The electronic circuitry receives power and clock from the alternating electric field. The electronic circuitry includes an integrated semiconductor circuitry, a pin hole, interchangeable analogue and digital sub-circuits, interchangeable opto-electronics sub-circuits, a MEMS mirror, integrated electrodes, a detector, and plurality of convertor units—to compute value of vital signs from the modulated optical spectrum and the sensed electrical charges.

### CROSS-REFERENCE TO RELATED APPLICATION

The present application is a continuation-in-part of a U.S. patent application Ser. No. 17/192,675 titled ‘A DEVICE FOR MEASURING OR STIMULATING VITAL SIGNS OF A USER’ filed on 2021 Mar. 04. The U.S. Ser. No. 17/192,675 further claims priority to U.S. Provisional Application No. 62/985,328, filed Mar. 5, 2020, which is incorporated herein by reference.

### BACKGROUND OF THE INVENTION

### 1. Field of the Invention

The present invention generally relates to a system for measuring vital signs of a user, and more particularly relates to a system for sensing state of an object in an environment.

### 2. Description of Related Art

Effective, efficient, and safe delivery of healthcare is dependent on the timely identification and treatment of a user's (also further called “patient”) condition. Failure to rescue patients in an early stage of physiological deterioration can result in permanent organ injury, extended medical treatment, increased recovery time, or death. These avoidable adverse events drive healthcare costs up and quality down.

In today's fast-paced and increasingly globalized market, the need for real-time tracking systems for objects has never been more critical to ensuring operational efficiency, transparency, and customer satisfaction. Tracking of objects is a critical component in modern supply chain management, enabling real-time visibility and control over the movement of goods.

By accurately monitoring each object's location, condition, and status, businesses can optimize delivery times, reduce the risk of errors, and improve inventory management. This capability is essential for ensuring timely deliveries, enhancing customer satisfaction, and enabling proactive response to disruptions or delays. As global trade continues to expand and consumer expectations rise, the need for robust tracking systems that integrate with various logistics and transportation platforms has become more pressing. Effective object tracking contributes to improved operational efficiency, reduced costs, and better compliance with regulatory standards across the supply chain.

Currently, environmental threats are increasing and thus there is a need to monitor the environment, harmful substances and their impacts on objects. IoT and sensors are now utilized to track goods/packets/parcel in a supply chain and also to monitor environmental threats. However, most of the available technology utilizes batteries and radio waves to communicate data—which is an extra burden to the environment and the users.

Therefore, there is a need for a system and method for sensing a state of an object. Further, the system and the method should be able to sense and communicate the state of the object wirelessly, battery free and radio-waves free to external devices.

### SUMMARY OF THE INVENTION

Disclosed is a system for sensing state of an object in an environment. The system includes a generator, an emitting electrode, a patch, and an electronic circuitry. The generator generates an alternating electric field that emits from the emitting electrode. The patch is in reach and receives the alternating electric field and the electronic circuitry is attached to the patch.

The patch is influenced by the alternating electric field and senses the state of the object and the environment. The patch has a top surface and a bottom surface. The patch includes a plurality of top markers, plurality of top control electrodes, a plurality of bottom markers, a plurality of bottom control electrodes, and a collector electrode.

The electronic circuitry includes an integrated semiconductor circuitry with a pin hole, a plurality of interchangeable analogue and digital sub-circuits, a plurality of interchangeable opto-electronics sub-circuits, a MEMS mirror, a plurality of integrated electrodes, a detector, and a plurality of convertor units.

The object's vital signs propagate through the objects and modulate the alternating electric field. The electronic circuitry computes the values of vital signs and the optical spectrum released from the object and the condition of the environment. Further, the electronic circuitry communicates the computed value via the alternating electric field for further processing.

An object of the present invention is to provide the electronic circuitry with a bottom electrode configured to attach on the collector electrode to receive modulated vital signs from the object as electrical charges. Further, the system includes a receiver unit to receive the computed value over the alternating electric field.

An object of the present invention is to provide the receiver unit with a receiving electrode, a receiver impedance unit, and a first electronically control switch. The receiving impedance unit is configured to change the computed value into voltage levels.

An object of the present invention is to provide the generator with a generator impedance unit for generating a controlled level of the alternating electric field. A second electronically control switch modulates the alternating electric field with instructions to arrange the plurality of interchangeable opto-electronics sub-circuits and the plurality of interchangeable analogue and digital sub-circuits.

Another object of the present invention is to provide the electronic circuitry with a computing unit configured to compute values of the vital signs from the modulated optical spectrum and the sensed electrical charges. Further, the computing unit communicates the computed values over the alternating electric field.

While a number of features are described herein with respect to embodiments of the inventions; features described with respect to a given embodiment also may be employed in connection with other embodiments. The following description and the annexed drawings set forth certain illustrative embodiments of the inventions. These embodiments are indicative, however, of but a few of the various ways in which the principles of the inventions may be employed. Other objects, advantages, and novel features according to aspects of the inventions will become apparent from the following detailed description when considered in conjunction with the drawings.

### BRIEF DESCRIPTION OF DRAWINGS

The annexed drawings, which are not necessarily to scale, show various aspects of the inventions in which similar reference numerals are used to indicate the same or similar parts in the various views.

FIG. 1 illustrates a schematic diagram of a system for sensing a state of an object;

FIG. 2 illustrates another schematic diagram of a patch interacting with a generator and a detector under influence of an alternating electric field; and

FIG. 3 illustrates another schematic diagram of the relationship between the generator, the patch and a receiver unit forming a closed electrical circuit.

### DETAILED DESCRIPTION OF DRAWINGS

The present disclosure is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

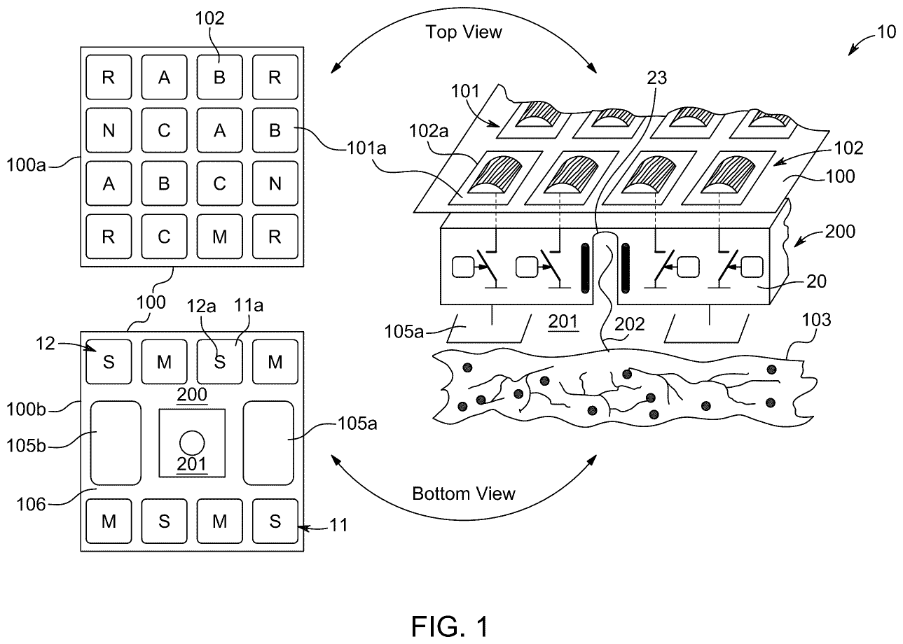

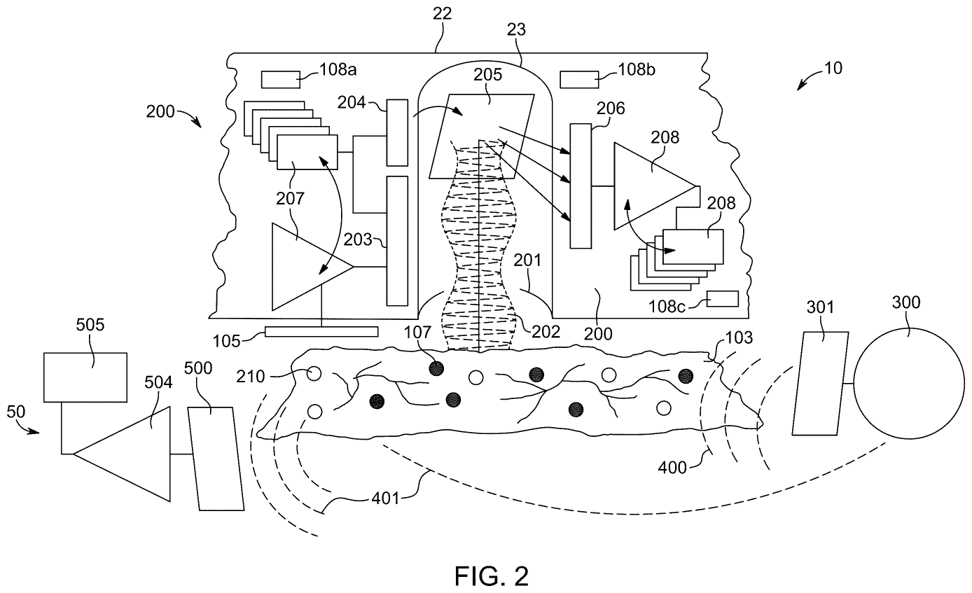

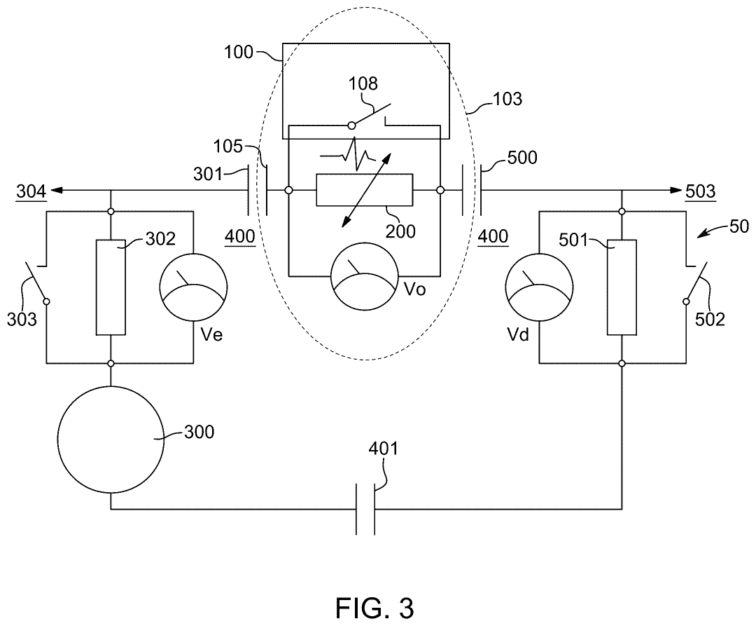

FIG. 1 illustrates a schematic diagram of a system 10 for sensing a state of an object 103 in an environment. The system 10 includes a generator (300, shown in FIG. 2), an emitting electrode (301, shown in FIG. 2), a patch 100, and an electronic circuitry 200. The generator 300 generates an alternating electric field. The emitting electrode 301 is configured to emit the generated alternating electric field from the generator 300 influencing the object. The generator 300 is explained in detail in conjunction with FIG. 3 of the present invention.

The patch 100 is configured to be positioned within reach of the object 103 and the alternating electric field. The patch 100 has a top surface 100a and a bottom surface 100b. The patch 100 includes a plurality of top markers 102, a plurality of top control electrodes 101, a plurality of bottom markers 12, a plurality of bottom control electrodes 101b and a collector electrode 106.

The plurality of top markers 102 are configured to attach on the top surface 100a of the patch 100. The plurality of top markers 102 are configured to sense the environment by influencing the alternating electric field. The plurality of top control electrodes 101, wherein a top control electrode 101a is configured to receive at least one top marker 102. Further, the top control electrode 101a controls flow of the alternating electric field from the respective top marker 102a.

The plurality of bottom markers 12 are configured to sense a state of the object 103 by influencing the alternating electric field. The plurality of bottom control electrodes 11, wherein at least one bottom control electrode 11a is configured to receive at least one bottom marker 12a. The bottom control electrode 11a controls flow of the alternating electric field from the respective bottom marker12a. In an embodiment, the top surface 100a and the bottom surface 100b is having a polymer foil which electrically insulates the top control electrodes 101 and the bottom control electrodes 11.

The plurality of top markers 102 and the plurality of bottom markers 12 are sensing elements that influences (e.g. changes) the alternating electric field to be measured, analysed and reported by the electronic circuitry 200. Examples of the influences include but not limited to impedance changes, building electrical charges, oscillating patterns—to identify changes in the environment/object.

Examples of the plurality of top markers 102 and the plurality of bottom markers 12 are biological markers (B) responding to bacterial or pathogen presence, atomic-level (A) markers responds to radiation (radioactive or thermal), chemical markers (C) responding to chemical contaminants, Noise/Blast markers (N) use piezoelectric materials to detect acoustic shock or explosion events, Structures (R) markers responding to detection of remote radio sources useful for EM warfare (locating enemy transmitters), and Miscellaneous (M) markers for detection of additional environmental, chemical, biological or physical phenomena.

The electronic circuitry 200 is configured to attach the patch 100. Further, the electronic circuitry 200 receives power and clock from the alternating electric field. The electronic circuitry 200 includes an integrated semiconductor circuitry 20, a pin hole 201, a MEMS mirror (205, shown in FIG. 2), a detector 206, and a computing unit (22, shown in FIG. 2).

The pin hole 201 is configured to be etched in the integrated semiconductor circuitry 20 to create an opening 23 to receive infrared signal 202 modulated with vital signs from the object 103. Examples of the infrared signal 202 received from the object 103 include but not limited to mechanical vibrations, bioelectric signals, biological processes, movements within the object 103, heat or thermal radiation, and electromagnetic fields.

The system 10 does not use cameras, lenses or illumination to measure vital signs or status of the object 103. The integrated semiconductor circuitry 20 is made up of silicon. The pin hole 201, the MEMS mirror 205, the detector (206, shown in FIG. 2 and FIG. 3) and the computing unit (22, shown in FIG. 2 and FIG. 3) are explained in detail in conjunction with FIG. 2 and FIG. 3 of the present invention.

The patch 100 is a modular platform that supports many reaction-based or property changing materials for multi-domain sensing. The plurality of top markers 102 and the plurality of bottom markers 12 operate in two fundamental ways i.e. receptive and reactive. Under receptive, the markers change a measurable property such as impedance, color and resistance.

Under reactive, the markers 102 and 12 generate an electric charge or potential that handles both passive sensing (detective changes) and active sensing (detecting self-generated signals). The sensing is a field-based sensing such as dielectric spectroscopy or electric-field tomography, etc.

The plurality of top control electrodes 101 and the plurality of bottom control electrodes 11 detects or influence the top makers 102 and the bottom markers 12, respectively through capactively coupling, bias voltage application, and grounding or isolation. The system 10 measures both static and dynamic electrical responses and probe materials by biasing them.

In one embodiment, the patch 100 further includes at least one bottom electrode 105a configured to attach on the collector electrode 106 to receive modulated vital signs from the object 103. For better results, one more bottom electrode 105b may be configured to attach on the collector electrode 106, together the bottom electrodes 105a, 105b surrounds the pin hole 201 for transmitting the received vital signs by modulating the alternating electric field to the processing unit (504, shown in FIG. 2) to enable bidirectional communication or control.

FIG. 2 illustrates another schematic diagram to illustrate a patch 100 interacting with a generator 300 and a detector 206 under influence of an alternating electric field. The MEMS mirror 205 is configured inside the opening 23 to reflect the modulated infrared signal received from the pin hole 201. Examples of the MEMS mirror 205 include but not limited to a beam splitter, phase detector, prism, polarizer, projector, oscillator or optical director, etc.

Further, the pin hole 201 is configured to be of a size of ~1 μm that admits or receives only near-field IR radiation from the object 103. The pin hole 201 acts as a physical wavelength filter and is able to detect heat-encoded biochemical and biophysical patterns of the object 103. The pin hole 201 is a nano-aperture IR sensor that captures short-range thermal emissions representing metabolic and physiological processes. In an embodiment, the pin hole 201 is etched in a circular shape. However, it will be readily apparent to those skilled in the art that the pin hole 201 may be envisioned in multiple shapes and sizes to support properties of the received infrared signals, without deviating from the scope of present invention.

Further, the MEMS mirror 205 creates an optical spectrum of the modulated infrared signal. The MEMS mirror 205 is a tiny, controllable mirror made using Micro-Electro-Mechanical Systems (MEMS) technology, essentially etched onto silicon to manipulate light beams for applications like projectors, barcode scanners, and LiDAR, offering fast, precise steering via electrostatic, electromagnetic, or piezoelectric drives.

The MEMS mirror 205 are miniature mirrors are key to compact optical systems, enabling functions such as 2D beam scanning and projection by tilting their surfaces in response to electrical signals, creating complex patterns or directing light precisely.

The detector 206 is configured to receive the reflected modulated optical spectrum and the sensed electrical charges to be transferred. The computing unit 22 is configured to compute the value of the vital signs from the modulated optical spectrum and the sensed electrical charges.

Examples of the detector 206 include but not limited to a photosensitive array, infrared photo diodes, transistors, opto-couplers, solar cells, temperature sensors, etc. Further, the computing unit 22 communicates the computed value over the alternating electric field. The computed value displays the status of the object 103. The computing unit 22 further includes a plurality of interchangeable analogue and digital sub-circuits 207, a plurality of interchangeable opto-electronics sub-circuits 208, a plurality of integrated electrodes 203, 204, and a plurality of convertor units 108a, 108b, 108c (hereinafter collectively used as 108 (shown in FIG. 3)).

The plurality of interchangeable analogue and digital sub-circuits 207 are arranged to process the modulated alternating electric field 202 to analyse the state of the object 103. The plurality of interchangeable opto-electronics sub-circuits 208 arranged to process the modulated infrared signals 202 to analyse the state of the object 103. Further, the interchangeable opto-electronics sub-circuits 208 compares the result to verify classification of analysis of the plurality of interchangeable analogue and digital sub-circuits 207.

The plurality of integrated electrodes 203, 204 are configured to surround the pin hole 201 to sense electrical charges in the alternating electric field 400 carried by the infrared signals 202. Further, at least one of the integrated electrode 204 manipulates the MEMS mirror 205 position to reflect the optical spectrum carrying the modulated alternating electric field 400. Alternatively, the integrated electrode 203 may also manipulate the MEMS mirror 205.

In an embodiment, the plurality of integrated electrodes 203, 204 are photosensitive region and is implemented as gate of a MOS transistor, are biased by the plurality of interchangeable analogue or digital subcircuits 207 to perform chopper functions, resonant field coupling or near-field manipulation.

The plurality of convertor units 108a, 108b, 108c are configured to convert values related to the state of the object 103 into a readable value. The readable value is converted from either analogue to digital values or digital to analogue values or analogue to optical values or digital to optical values or optical to analogue values.

Further, the plurality of convertor units 108a, 108b, 108c modulate the values of the analysis of the plurality of interchangeable opto-electronics sub-circuits 208 and the plurality of interchangeable analogue and digital sub-circuits 207 to be communicated over the alternating electric field. Furthermore, the plurality of convertor units 108a, 108b, 108c manages flow/measurement of the alternating electric field received from the collector electrode 106 through the plurality of markers 102, the plurality of top control electrodes 101 and the plurality of bottom control electrodes 11.

The plurality of interchangeable opto-electronics sub-circuits 208 and the plurality of interchangeable analogue and digital sub-circuits 207 computes the value of the vital signs from the modulated optical spectrum and the sensed electrical charges to be communicated over a communication network.

Examples of the interchangeable analogue sub-circuits 207 include but not limited to amplifiers, attenuators, comparators, logarithmic, exponential convertors, integrators, differentiators, adders, and similar structures. Examples of the interchangeable digital sub-circuits 208 include but not limited interchangeable arrangement of logic gates, FPGA-like fabrics, counters, shift registers, flip-flops, memory structures, arithmetic units, etc.

Examples of the interchangeable opto-electronics sub-circuits 208 include but not limited to interchangeable arrangement of photodetectors, light guides, phase arrays, light switches, MEMS mirrors, optical spectrum or phase analysers, lasers, other integrated light sources, etc. It will be apparently skilled to those skilled in the art various number of sub-circuits 207 and 208 may be envisioned depending on high-integration semiconductor processes without deviating from the scope of the present invention.

The system 10 further includes a receiver unit 50 configured to receive the computed value from the computing unit 22 through the alternating electric field 401. The alternating electric field 400 is modulated with the infrared signals or vital signs, and is converted to the alternating electric field 401 containing computed values of the infrared signals with vital signs.

The receiver unit 50 is explained in detail in conjunction with FIG. 3 of the present invention. The receiver unit 50 communicates the computed value to a processing unit 504 for further processing. Further, the processing unit 504 is able to communicate computed values with external networks over the communication network via an interface unit 505.

The object 103 is either a living entity (e.g. humans, animals, plants) or a non-living entity (e.g. machines, vehicles, robots, weapons, drones, telecom apparatus, etc.). The patch 100 is configured to attach with object 103 surface (e.g. skin, hull, metal frame, etc.).

For example, the system 10 is able to compute value like vital signs of the living body of the object 103 such as ECG, EEG, myogram (EMG), inflammation, allergic reactions, organ activities (e.g. stones in gall bladder or kidneys), etc. Similarly, the system 10 is able to locate position, condition, movements and quality of the object 103.

The plurality of convertor units 108a, 108b, 108c provide interchangeable routing and/or mixing of analogue, digital and optoelectronic information. The combination of electronic and optoelectronic processing paths is possible. The arrangement and interconnection of these sub-circuits 207, 208 are controlled by the processing unit 504. The system 10 receives power and clock from the alternating electric field 400 generated by the generator 300 to supply power to the integrated semiconductor circuitry 20 and its frequency serves as the timing reference for internal sub-circuits.

FIG. 3 illustrates another schematic diagram to illustrate circuit relationship of the generator 300, electronic circuitry 200 and the receiver unit 50. The receiver unit 50 further includes a receiving electrode 500, a receiver impedance unit 501, and a first electronically control switch 502.

The receiving electrode 500 is configured to transmit received computed value via the alternating electric field 401 and closes alternating circuit with the generator 300. The receiver impedance unit 501 is configured to receive the transmitted computed value from the receiving electrode 501. Further, the receiver impedance unit 501 changes the computed value into voltage levels.

The first electronically control switch 502 is configured to bi-directional communicate instructions with the electronic circuitry 200 and the generator 300. The first electronically control switch 502 receives instructions over a communication network. The modulations in the alternating electric field 400 are measurable at control points 304 and 503 connected to external electronics (not shown in FIGURES) through the interface unit (505, shown in FIG. 2). The measurement of modulations allows detection of analogue signals, extraction of biometrics, and real-time analytics or machine-learning-based classification.

The generator 300 further includes a generator impedance unit 302, and a second electronically control switch 303. The generator impedance unit 302 generates controlled level of the alternating electric field. The second electronically control switch 303 modulates the alternating electric field with instructions to arrange the plurality of interchangeable opto-electronics sub-circuits 208 and the plurality of interchangeable analogue and digital sub-circuits 207. The second electronically control switch 503 receives instructions over a communication network.

Examples of the generator 300 include but not limited to hardware or software oscillators, RC oscillators, quartz oscillators, Temperature Compensated Crystal Oscillator (TCXO), surface acoustic wave (SAW), etc. Examples of the generator impedance unit 302 include but not limited to inductors, capacitors, resistors, transistors, diodes, filters, etc. Examples of the first electronically control switch 502 and the second electronically control switch 303 include but not limited to IO ports, MOS transistors, opto couplers, relays, and logic gates.

Following is an example of voltage distribution in a serial AC network between the patch, the object 103 and the electronic circuitry 200. The generator impedance unit 302 generates a RMS voltage (Ve—Voltage emitter) of 5V from the oscillation frequency of 500 kHz received from the generator 300.

If impedances from the electronic circuitry 200 and the receiving impedance unit 501 are equal to the impedance from the generator impedance unit 302 then by Kirchhoff's voltage law—the electronic circuitry 200 (Vo—Voltage Object) and the receiving impedance unit 501 (Vd—Voltage detector) also exhibits 5V RMS in the serial chain.

As shown in FIG. 3, the entire circuit is capacitively coupled back from the receiving impedance unit 501 to the generator 300 via the same electric field 401, enabling contactless and wireless operation over distances at least 50 cm.

Following is an example of data modulation through electronic switching: Closing one of the electronically control switches (303, 502) and the convertor unit (108) to effectively bypass the respective impedances generated from the generator impedance unit 302, the receiver impedance unit 501 and impedance of the electronic circuitry 200 results in OV over the closed switch while the remaining impedance units rise each to 7.5V to establish bi-directional data communication between each other. Re-opening the electronically control switches (303, 502), and the convertor unit (108) restore the original 5-5-5 voltage distribution.

Through the data modulation, the generator 300 transmits data to the patch 100 utilizing the processing unit (504, shown in FIG. 2), the electronically switches (303, 502) and the convertor unit (108). The electronic circuitry 200 extracts system clock from the alternating electric field frequency to process the data, while the alternating electric field is also converted into power (e.g. 5V) to provide energy to the electronic circuitry 200.

The impedance from the electronic circuitry 200 is additionally influenced by electric-field waves modulated with biochemical activity, mechanical vibration, thermal infrared radiation emitted by the object 103 and environmental radiations. These phenomena induce a complex (“cacophonic”) modulation signature resulting from molecular, atomic, and electron-level motion. These complex modulations are identified, monitored, classified and computed to measure the physical and mental state of the object 103 as well as the environment using the system (10, shown in FIG. 1).

In one embodiment, a patch includes a top marker, a top control electrode, a bottom marker, a bottom control electrode, and a control electrode. The top marker is configured to attach on the top surface of the patch to sense the environment by influencing the alternating electric field.

The top control electrode is configured to receive the top marker to control flow of the alternating electric field from the respective top marker. The bottom marker is configured to sense state of the object by influencing the alternating electric field. The bottom control electrode is configured to receive the bottom marker to control flow of the alternating electric field from the respective bottom marker.

The collector electrode is configured to attach to the bottom surface of the patch. Further, the collector electrode is configured to capactively couple with the bottom marker, the bottom control electrode, the top marker and the top control electrode-to receive the alternating electric field through the top marker and the bottom marker;

Exemplary use cases of the system are as follows:

Healthcare: Patients/Elderly/children/maternal are to be monitored in any situation by attaching the invention related patch near their body or medical equipment (e.g. infusion bags, catheters, etc.) to measure their vital signs, diseases and healing process with a battery-free and radio waves free patch.

Defence: Soldiers or machines like (UAV, drones, torpedoes) detects harmful substances or radiation in the environment, as well as the impact on their body or constitution. Further, the patch is able to detect hostile manipulation of the environment, and jamming signals as well as radio transmitters—may be carried by drones or other UAV.

Food Quality: The patch detects quality of food and water, as well as allergic reactions from the living object.

Agritech: The patch is able to control the growth and well-being of trees and plants, e.g. bacterial infestation, mould, fire, contamination of water or air. The patch is able to check their reaction to the environment and communicate with responders. Variations of the patch may also be submerged into liquids to see changes or concentrations of substances of interest. The patch may also be applied to compartments like food storage, livestock, housing, fridges, trucks, trailers, etc. to monitor or assure necessary environmental conditions.

There has thus been outlined, rather broadly, the more important features of the disclosure in order that the detailed description thereof may be better understood, and in order that the present contribution to the art may be better appreciated. There are additional features of the disclosure that will be described hereinafter and which will also form the subject matter of the claims appended hereto. The features listed herein and other features, aspects and advantages of the present disclosure will become better understood with reference to the following description and appended claims.

Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

1. A system for sensing a state of an object in an environment, the system comprising:
a generator to generate an alternating electric field;
an emitting electrode configured to emit the generated alternating electric field from the generator influencing the object;
a patch configured to be positioned in reach of the object and the alternating electric field, the patch having a top surface and a bottom surface, the patch comprising:
a plurality of top markers configured to attach to the top surface of the patch, the plurality of top markers configured to sense the environment by influencing the alternating electric field;
a plurality of top control electrodes, wherein at least one top control electrode is configured to receive at least one top marker, further each of the top control electrode controls flow of the alternating electric field from the respective top marker;
a plurality of bottom markers configured to sense a state of the object by influencing the alternating electric field;
a plurality of bottom control electrodes, wherein at least one bottom control electrode is configured to receive at least one bottom marker, further each of the bottom control electrode controls flow of the alternating electric field through the respective bottom marker; and
a collector electrode configured to attach to the bottom surface of the patch, further the collector electrode configured to capacitively couple with the bottom markers, the bottom control electrodes, the top markers and the top control electrodes to receive the alternating electric field through the top markers and the bottom markers;
an electronic circuitry configured to attach to the patch, further the electronic circuitry receives power and clock from the alternating electric field, the electronic circuitry comprising:
an integrated semiconductor circuitry;
a pin hole configured to be etched in the integrated semiconductor circuitry to create an opening to receive an infrared signal modulated with vital signs from the object;
a plurality of interchangeable analogue and digital sub-circuits arranged to process the modulated alternating electric field from the object to create analogue and digital values representing the state of the object;
a plurality of interchangeable opto-electronics sub-circuits arranged to process the modulated infrared signals from the object to create optical values representing the state of the object, further the interchangeable opto-electronics sub-circuits compare the values to verify a classification of analysis of the plurality of interchangeable analogue and digital sub-circuits;
a MEMS mirror configured inside the opening to reflect the modulated infrared signal received from the pin hole, further the MEMS mirror creates an optical spectrum of the modulated infrared signal;
plurality of integrated electrodes configured to surround the pin hole to sense electrical charges in the alternating electric field carried by the infrared signals, further at least one of the integrated electrode manipulates a position of the MEMS mirror;
a detector configured to receive the reflected modulated optical spectrum and the sensed electrical charges to be transferred;
plurality of convertor units to convert values related to the state of the object into at least one of: analogue to digital values; digital to analogue values; analogue to optical values; digital to optical values; and optical to analogue values, and further the plurality of convertor units modulate the results of the analysis of the plurality of interchangeable opto-electronics sub-circuits and the plurality of interchangeable analogue and digital sub-circuits to be communicated over the alternating electric field;
wherein the plurality of interchangeable opto-electronics sub-circuits and the plurality of interchangeable analogue and digital sub-circuits computes the value of the vital signs from the modulated optical spectrum and the sensed electrical charges.

2. The system according to claim 1, wherein the object comprises at least one of: a living entity; a non-living entity.

3. The patch according to claim 1 further comprising at least one bottom electrode configured to attach to the collector electrode to transmit received vital signs by modulating the alternating electric field.

4. The system according to claim 1 further comprising a receiver unit configured to receive the computed value over the alternating electric field.

5. The receiver unit according to claim 4 further comprising:
a receiving electrode configured to transmit the computed value via the alternating electric field;
a receiver impedance unit configured to receive the transmitted computed value from the receiving electrode, further the receiver impedance unit changes the computed value into voltage levels; and
a first electronically control switch configured to bi-directional communicate instructions with the electronic circuitry and the generator, wherein the first electronically control switch receives instructions over a communication network.

6. The generator according to claim 1 comprising:
a generator impedance unit for generating controlled level of the alternating electric field; and
a second electronically control switch to modulate the alternating electric field with instructions to arrange the plurality of interchangeable opto-electronics sub-circuits and the plurality of interchangeable analogue and digital sub-circuits, wherein the second electronically control switch receives instructions over a communication network.

7. A system for sensing a state of an object in an environment, the system comprising:
a generator to generate an alternating electric field;
an emitting electrode configured to emit the generated alternating electric field from the generator for influencing the object;
a patch configured to position in reach of the object and the alternating electric field, the patch having a top surface and a bottom surface, the patch comprising:
a plurality of top markers configured to attach on the top surface of the patch, the plurality of top markers configured to sense the environment by influencing the alternating electric field;
a plurality of top control electrodes, wherein at least one top control electrode is configured to receive at least one top marker, further each of the top control electrode controls flow of the alternating electric field through the respective top marker;
a plurality of bottom markers configured to sense a state of the object by influencing the alternating electric field;
a plurality of bottom control electrodes, wherein at least one bottom control electrode is configured to receive at least one bottom marker, further each of the bottom control electrode controls flow of the alternating electric field through the respective bottom marker; and
a collector electrode configured to attach to the bottom surface of the patch, further the collector electrode configured to capactively couple with the bottom markers, the bottom control electrodes, the top markers and the top control electrodes to receive the alternating electric field through the top markers and the bottom markers;
an electronic circuitry configured to attach to the patch, further the electronic circuitry receives power and clock from the alternating electric field, the electronic circuitry comprising:
an integrated semiconductor circuitry;
a pin hole configured to be etched in the integrated semiconductor circuitry to create an opening to receive infrared signal modulated with vital signs from the object;
a MEMS mirror configured inside the opening to reflect the modulated infrared signal received from the pin hole, further the MEMS mirror creates an optical spectrum of the modulated infrared signal;
a detector configured to receive the reflected modulated optical spectrum and the sensed electrical charges to be transferred; and
a computing unit configured to compute value of the vital signs from the modulated optical spectrum and the sensed electrical charges, further the computing unit communicates the computed value over the alternating electric field.

8. The computing unit according to claim 1 further comprising:
a plurality of interchangeable analogue and digital sub-circuits arranged to process the modulated alternating electric field from the object to create analogue and digital values representing the state of the object;
a plurality of interchangeable opto-electronics sub-circuits arranged to process the modulated infrared signals from the object to create optical values representing the state of the object, further the interchangeable opto-electronics sub-circuits compares the values to verify classification of analysis of the plurality of interchangeable analogue and digital sub-circuits;
a plurality of integrated electrodes configured to surround the pin hole to sense electrical charges in the alternating electric field carried by the infrared signals, further at least one of the integrated electrode manipulates position of the MEMS mirror; and
a plurality of convertor units configured to convert values related to the state of the object into at least one of: analogue to digital values; digital to analogue values; analogue to optical values; digital to optical values; and optical to analogue values, and further the plurality of convertor units modulate the values of the analysis of the plurality of interchangeable opto-electronics sub-circuits and the plurality of interchangeable analogue and digital sub-circuits with the alternating electric field;
wherein the plurality of interchangeable opto-electronics sub-circuits and the plurality of interchangeable analogue and digital sub-circuits computes the value of the vital signs from the modulated optical spectrum and the sensed electrical charges to be communicated over a communication network.

9. The patch according to claim 7 further comprising at least one bottom electrode configured to attach on the collector electrode to transmit the received vital signs by modulating the alternating electric field.

10. The system according to claim 7 further comprising a receiver unit configured to receive the computed value over the alternating electric field.

11. The receiver unit according to claim 10 further comprising:
a receiving electrode configured to transmit received computed value via the alternating electric field;
a receiver impedance unit configured to receive the transmitted computed value from the receiving electrode, further the receiver impedance unit changes the computed value into voltage levels; and
a first electronically control switch configured to bi-directional communicate instructions with the electronic circuitry and the generator, wherein the first electronically control switch receives instructions over a communication network.

12. The generator according to claim 7 comprising:
a generator impedance unit for generating controlled level of the alternating electric field; and
a second electronically control switch to modulate the alternating electric field with instructions to arrange the plurality of interchangeable opto-electronics sub-circuits and the plurality of interchangeable analogue and digital sub-circuits, wherein the second electronically control switch receives instructions over the communication network.

13. A system for sensing a state of an object in an environment, the system comprising:
a generator to generate an alternating electric field;
an emitting electrode configured to emit the generated alternating electric field from the generator influencing the object;
a patch configured to position in reach of the object and the alternating electric field, the patch having a top surface and a bottom surface, the patch comprising:
a top marker configured to attach on the top surface of the patch to sense the environment by influencing the alternating electric field;
a top control electrode configured to receive the top marker to control flow of the alternating electric field from the respective top marker;
a bottom marker configured to sense the state of the object by influencing the alternating electric field;
a bottom control electrode configured to receive the bottom marker to control flow of the alternating electric field from the respective bottom marker;
a collector electrode configured to attach to the bottom surface of the patch, further the collector electrode configured to capactively couple with the bottom marker, the bottom control electrode, the top marker and the top control electrode to receive the alternating electric field through the top marker and the bottom marker;
an electronic circuitry configured to attach to the patch, further the electronic circuitry receives power and clock from the alternating electric field, the electronic circuitry comprising:
an integrated semiconductor circuitry;
a pin hole configured to be etched in the integrated semiconductor circuitry to create an opening to receive infrared signal modulated with vital signs from the object;
a MEMS mirror configured inside the opening to reflect the modulated infrared signal received from the pin hole, further the MEMS mirror creates an optical spectrum of the modulated infrared signal;
a detector configured to receive the reflected modulated optical spectrum and the sensed electrical charges to be transferred; and
a computing unit configured to compute the value of the vital signs from the modulated optical spectrum and the sensed electrical charges, further the computing unit communicates the computed value over the alternating electric field.
